(19) United States
(12) Patent Application Publication
(10) Pub. No.: US 2025/0249143 A1
(43) Pub. Date: Aug. 7, 2025

(54) SYNTHETIC BONE GRAFT AND METHOD FOR USING SAME

(71) Applicant: SurGenTec, LLC, Boca Raton, FL (US)

(72) Inventors: Travis Greenhalgh, Boca Raton, FL (US); Guilherme Pires, Boca Raton, FL (US)

(21) Appl. No.: 19/047,054

(22) Filed: Feb. 6, 2025

Related U.S. Application Data
(60) Provisional application No. 63/550,317, filed on Feb. 6, 2024.
(60) Provisional application No. 63/745,640, filed on Jan. 15, 2025.

(51) Int. Cl. A61L 27/12; A61L 27/10; A61L 27/20; A61L 27/24

(52) U.S. Cl. CPC A61L 27/12; A61L 27/10; A61L 27/20; A61L 27/24; A61L 2400/06; A61L 2430/02

(57) ABSTRACT

A synthetic, hydrophilic bone graft can be formed with carbonate apatite, methylcellulose, Bioglass, and collagen. The bone graft material can be hydrated in an aqueous solution to turn it into a viscoelastic material. The bone graft can then be injected to a target site to improve bone healing and revascularize a target area. The viscoelastic form can allow the bone graft to remain cohesive while fitting through small apertures.

### PRIORITY

This application claims the benefit of priority under 35 U.S.C. § 119 (e) to U.S. Provisional Patent Application No. 63/550,317, filed Feb. 6, 2024, and U.S. Provisional Patent Application No. 63/745,640, filed Jan. 15, 2025. Both of these applications are hereby incorporated by reference herein in their entireties. Any and all applications for which a foreign or domestic priority claim is identified in the Application Data Sheet as filed with the present application are hereby incorporated by reference under 37 CFR 1.57.

### BACKGROUND

### Field

The present application relates to orthopedic surgery in general, spine, bone graft delivery systems and methods, and more particularly synthetic bone graft material.

### Description of the Related Art

In a bone grafting procedure, a surgeon places bone or a bone substitute into an area in a patient's body to provide a type of scaffold for bone growth and repair. Bone grafts can be used to help treat various orthopedic problems, for example, to fuse a joint or repair a fracture. Bone graft material can be, for example, autogenous (harvested from the patient's own body), allogeneic (harvested from another person, usually a cadaver), or synthetic. Many bone grafting procedures are performed via open surgery implantation. However, these procedures can also be performed minimally invasively, for example, by using a bone graft delivery system to deliver bone graft material into the target location without requiring a large surgical incision.

In some cases decortication of the bony area receiving the graft is performed prior to delivery of the bone graft material. Decortication removes superficial cortical bone and exposes the underlying cancellous bone, which can help accelerate the integration of the bone graft with the native bone.

Synthetic bone grafts known in the art are often hydrophobic. Such bone grafts may wick up mesenchymal cells, bone morphogenic proteins, and other synthetic or natural growth factors that enhance the opportunity for fusion. Other bone grafts in the field often contain carriers that make for a hydrophobic product, such as porcine gel or glycerin. Other bone grafts in the field often lack moldability and flowability. Therefore, there is a need for bone grafts with improved properties.

### SUMMARY

The devices, compositions, systems, and methods described herein relate to a synthetic bone graft that may be reconstituted in liquid.

In some embodiments, the methods described herein relate to forming a bone graft, the method including: mixing carbonate apatite, methylcellulose, Bioglass, and collagen to form a bone graft material, wherein the methylcellulose has a molecular weight of greater than 500; sterilizing the bone graft material; mixing the bone graft material with an aqueous solution; filling a device with the bone graft material; and inserting, using the device, the bone graft material into a patient such that it contacts a bone of the patient, wherein the bone graft material is viscoelastic, and wherein the bone graft material flows through a distal end of the device to contact the bone.

In some embodiments, sterilizing the bone graft material includes sterilizing the bone graft material using gamma radiation. In some embodiments, sterilizing the bone graft material includes sterilizing the bone graft material using electron beam. In some embodiments, the aqueous solution includes at least one of blood, saline, bone marrow aspirate, or stem cells. In some embodiments, the carbonate apatite has a particle size between 0.3 microns and 1 micron. In some embodiments, the carbonate apatite has a particle size between 0.3 microns and 2 microns. In some embodiments, the methylcellulose has a molecular weight of between 650 and 750. In some embodiments, the distal end of the device includes a nozzle. In some embodiments, the nozzle has a diameter of 1-5 mm. In some embodiments, the collagen is fibrillar collagen. In some embodiments, the collagen is not lyophilized. In some embodiments, inserting the bone graft material into the patient includes inserting a rasp into an incision and delivering the bone graft material into the incision.

In some embodiments, the compositions described herein relate to a bone graft including carbonate apatite; methylcellulose with a molecular weight of greater than 500; Bioglass; and fibrillar collagen, wherein the composition is viscoelastic, wherein the composition is configured to flow through a distal end of a device to a target area, and wherein the composition is configured to revascularize the target area.

In some embodiments, the carbonate apatite has a particle size between 0.3 microns and 1 micron. In some embodiments, the carbonate apatite has a particle size between 0.3 microns and 2 microns. In some embodiments, the methylcellulose has a molecular weight of between 650 and 750. In some embodiments, the collagen is not lyophilized. In some embodiments, the carbonate apatite, methylcellulose, and Bioglass are embedded in the collagen.

In some examples, the bone graft material flows through a nozzle of the device. In some examples, the composition is about 5-15% fibrillar collagen, about 5-20% methylcellulose, about 0.5-10% Bioglass, and about 60-90% carbonate apatite.

In some examples, methods for using a bone graft described herein can include: providing a bone graft material including calcium phosphate, methylcellulose, Bioglass, and collagen; mixing the bone graft material with an aqueous solution; and inserting the bone graft material into a patient such that it regenerates a bone of the patient.

In some examples, the methylcellulose has a molecular weight of greater than 500. In some examples, the bone graft material is sterilized. In some examples, the bone graft material is viscoelastic. In some examples, the bone graft material flows through a distal end of a device to contact the bone. In some examples, the calcium phosphate includes carbonate apatite. In some examples, the aqueous solution includes at least one of blood, saline, bone marrow aspirate, or stem cells. In some examples, the carbonate apatite has a particle size between 0.3 microns and 1 micron. In some examples, the carbonate apatite has a particle size between 0.3 microns and 2 microns. In some examples, the methylcellulose has a molecular weight of between 650 and 750. In some examples, the bone graft material flows through a nozzle of the device, the nozzle having a diameter of 1-5 mm. In some examples, inserting the bone graft material into the patient includes: inserting a rasp into an incision; and delivering the bone graft material into the incision. In some examples, the methylcellulose has a viscosity of between 5,000 cPs and 15,000 cPs.

In some examples, compositions for a bone graft described herein can include: calcium phosphate; methylcellulose; Bioglass; and fibrillar collagen, wherein the fibrillar collagen is not lyophilized, and wherein the fibrillar collagen includes loose fibers, wherein the composition is configured to regenerate bone.

In some examples, the calcium phosphate is carbonate apatite. In some examples, the methylcellulose has a molecular weight of greater than 500. In some examples, the composition is viscoelastic. In some examples, the composition is configured to flow through a distal end of a device to a target area. In some examples, the carbonate apatite has a particle size between 0.3 microns and 1 micron. In some examples, the carbonate apatite has a particle size between 0.3 microns and 2 microns. In some examples, the methylcellulose has a molecular weight of between 650 and 750. In some examples, the carbonate apatite, methylcellulose, and Bioglass are embedded in the fibrillar collagen. In some examples, the composition expands at least 0.05 mm when hydrated. In some examples, the composition exerts less than 1.5 N of force on surrounding structures during expansion. In some examples, the composition expands at least 0.35 mm when hydrated. In some examples, the composition is about 5-15% fibrillar collagen, about 5-20% methylcellulose, about 0.5-10% Bioglass, and about 60-90% carbonate apatite. In some examples, the methylcellulose has a viscosity of between 5,000 cPs and 15,000 cPs.

In some embodiments, the techniques described herein relate to an apparatus substantially as shown and/or described. In some embodiments, the techniques described herein relate to a method substantially as shown and/or described. In some embodiments, the techniques described herein relate to a system substantially as shown and/or described.

### BRIEF DESCRIPTION OF THE DRAWINGS

FIG. 1 illustrates an example of a method for forming and using bone graft material.

FIG. 2 illustrates an example of bone graft material with collagen.

FIG. 3A illustrates an example of bone graft material with collagen before sterilization.

FIG. 3B illustrates an example of the bone graft material with the collagen of FIG. 3A after sterilization.

FIG. 3C illustrates another example of the bone graft material with the collagen of FIG. 3A after sterilization.

FIG. 3D illustrates an example of the bone graft material with collagen of FIG. 3A after sterilization and during mixing with aqueous solution.

FIG. 3E illustrates an example of the bone graft material with collagen of FIG. 3A after sterilization and after mixing with aqueous solution.

FIG. 3F illustrates an example of the bone graft material with collagen of FIG. 3A after being sterilized, mixed with aqueous solution, and formed into a ball.

FIG. 4 illustrates an example of bone graft material being pushed through a nozzle of a device.

FIG. 5A illustrates an example of collagen before sterilization.

FIG. 5B illustrates an example of the collagen of FIG. 5A after sterilization.

FIGS. 6A-B illustrate an example of a sagittal cut CT scan of the bone graft material with collagen after posterolateral fusion.

FIGS. 7A-B illustrate an example of a coronal view CT scan of the bone graft material with collagen after posterolateral fusion.

FIGS. 8A-B illustrate an example of a CT scan of the bone graft material without collagen after posterolateral fusion.

FIG. 9A shows an example of bone graft material in an aqueous solution.

FIG. 9B shows the example of the bone graft material of FIG. 9A compacted into a ball.

FIG. 9C shows the example of the bone graft material of FIG. 9A in an expanded state.

FIG. 9D shows the example of the bone graft material of FIG. 9A being pulled in different directions.

FIG. 10A shows an example of bone graft material in an posterior lumbar interbody fusion (PLIF) cage.

FIG. 10B shows the example of bone graft material in the PLIF cage of FIG. 10A at a first time.

FIG. 10C shows the example of bone graft material in the PLIF cage of FIG. 10A at a second time.

FIG. 10D shows the example of bone graft material in the PLIF cage of FIG. 10A at a third time.

FIG. 11A shows an example of bone graft material in an anterior lumbar interbody fusion (ALIF) cage at a first time.

FIG. 11B shows the example of bone graft material in the ALIF cage of FIG. 11A at a second time.

FIG. 11C shows the example of bone graft material in the ALIF cage of FIG. 11A at a third time.

FIG. 12A shows an embodiment of bone graft material in an ALIF cage in a test apparatus.

FIG. 12B is a graph of the displacement over time of an embodiment of the bone graft material described herein.

FIG. 12C is a graph of the force exerted by an embodiment of the bone graft material described herein over time.

FIG. 13 shows an example of a cage positioned between vertebrae.

FIG. 14 shows another example of a cage positioned between vertebrae.

FIG. 15 shows a 3 month pre-clinical testing CT scan of a solid posterior lateral fusion in an animal model using an example of bone graft material 1500.

### DETAILED DESCRIPTION

In certain examples, the bone grafts described herein can be hydrophilic bone grafts or other suitable types of bone grafts that may be reconstituted in a liquid. The bone graft can be synthetic. In examples, the bone graft material described herein may be viscoelastic, allowing it to flow through small apertures and remain cohesive in the body of a patient, preventing migration.

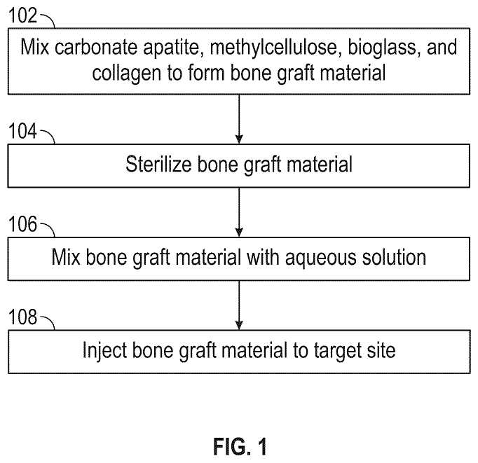

FIG. 1 illustrates an example of a method for forming and using bone graft material.

As shown at block 102, the bone graft material described herein can be a composition formed by mixing carbonate apatite, methylcellulose, Bioglass, and/or collagen. These materials can be placed into a vial or container. A kit associated with the material may contain a spoon or spatula to make it easy for a user to mix the graft with a fluid. In some embodiments, the materials can be dry when placed into the vial or container. In some embodiments, the carbonate apatite and methylcellulose can be mixed before the collagen is mixed in. In some examples, this can allow the collagen to be more evenly embedded with carbonate apatite and methylcellulose.

In certain examples, the bone graft material may include calcium phosphate, for example carbonate apatite or calcium carbonate, and Bioglass, such as the lowest amount of Bioglass possible by weight. In some examples, the bone graft material can be approximately 7.7% collagen, approximately 11.5% methylcellulose, approximately 2.88% Bioglass, and approximately 77.92% carbonate apatite. In some examples, the bone graft material can be 7-8% collagen, 11-12% methylcellulose, 2-3% Bioglass, and 77-78% carbonate apatite. In some examples, the bone graft material can be 5-15% collagen, 5-20% methylcellulose, 0.5-10% Bioglass, and 60-90% carbonate apatite. In some examples, the bone graft material can include approximately 70-90% carbonate apatite, approximately 5-15% methylcellulose, approximately 1-7% Bioglass, and approximately 5-20% collagen by weight. In some embodiments, the bone graft material can include approximately 75% carbonate apatite, approximately 10% methylcellulose, approximately 5% Bioglass, and approximately 10% collagen by weight. In some embodiments, the bone graft material can include approximately 50-95% carbonate apatite, approximately 1-30% methylcellulose, approximately 0.1-20% Bioglass, and approximately 1-50% collagen by weight.

In some embodiments, the bone graft material can not include collagen, which can decrease cohesiveness and increase migration in the body, but this may suffice for certain cases such as when the bone graft material is held in place in a bone void. In these examples, the bone graft material can include approximately 70-90% carbonate apatite, approximately 10-30% methylcellulose, and approximately 1-7% Bioglass by weight.

The bone graft material described herein can include carbonate apatite. A composite of calcium carbonate and hydroxyapatite can yield carbonate apatite. In some examples, the bone graft material can include a calcium phosphate including but not limited to carbonate apatite. In some examples, the calcium phosphate can be carbonate apatite, hydroxyapatite, calcium carbonate, monocalcium phosphate, dicalcium phosphate, tricalcium phosphate, tetracalcium phosphate, octacalcium phosphate, and/or an amorphous calcium phosphate. In some embodiments, calcium carbonate can be used instead of or in addition to carbonate apatite. The carbonate apatite can have a particle size small enough to access apertures or spaces for filling. Advantageously, carbonate apatite particles of a smaller size can allow for superior handling and flowability. Smaller particles of carbonate apatite can also improve porosity for optimal bone growth. Smaller particles may contain pore sizes within the particle ranging from 100-350 microns to optimize vascular ingrowth and bone regeneration. Average pore size can range from 100-250 microns, such as 150-200 microns or around 175 microns. The overall interconnected porosity may be between 65 and 90 percent, such as around 75 to 80 percent. In some embodiments, the overall interconnected porosity may be between 70 and 88 percent, such as around 77-81 percent.

The carbonate apatite can have a particle size between approximately 0.3 microns and approximately 1 micron. In some embodiments, the carbonate apatite can have a particle size between approximately 0.3 microns and approximately 2 microns. In some embodiments, the carbonate apatite can have a particle size between approximately 0.1 microns and approximately 3 microns. In some embodiments, the carbonate apatite can have a particle size between approximately 0.1 microns and approximately 5 microns. In some embodiments, the carbonate apatite can have a particle size of approximately 0.5 microns.

The bone graft material described herein can include methylcellulose. Methylcellulose can increase the handling and hydrophilic properties of the bone graft material. This can be advantageous for promoting revascularization and healing. Methylcellulose can be bioresorbable and only remain in the body for a short period of time. Methylcellulose, for example carboxymethylcellulose, can act as a binding agent or adhesive. Methylcellulose can bind the collagen together when the bone graft material is mixed with water. The methylcellulose can bind other particles to the collagen. Advantageously, using methylcellulose can allow the collagen to adhere to particles even when the collagen is not lyophilized. The loose collagen can adhere to bone marrow aspirate at the target site without being lyophilized to a sponge-like form due to the binding properties of the methylcellulose.

In some embodiments, the methylcellulose can be sodium carboxymethyl cellulose or carboxymethylcellulose sodium salt. The degree of substitution of methylcellulose can be defined as the average number of methoxyl groups attached to each of the anhydroglucose units along the chain. The degree of substitution of the methylcellulose can be degree of substitution is between 0.9 and 1.2. In some embodiments, the degree of substitution of the methylcellulose can be degree of substitution is between 0.5 and 1.5. Advantageously, this degree of substitution can enhance the material's viscosity for handling and adherence to irregular bone surfaces. In some examples, the viscosity of the methylcellulose can be between 4,000 cPs and 8,000 cPs. In some examples, the viscosity of the methylcellulose can be between 5,000 cPs and 15,000 cPs. In some examples, the viscosity of the methylcellulose can be between 8,000 cPs and 9,000 cPs. In some examples, the viscosity of the methylcellulose can be between 3,000 cPs and 15,000 cPs. In some examples, the viscosity of the methylcellulose can be between 2,000 cPs and 20,000 cPs. In some examples, the viscosity of the methylcellulose can be between 1,000 cPs and 25,000 cPs. The viscosity can be high enough to resist crosslinking during sterilization. This can also enhance the biodegradability and biocompatibility to foster long-term growth of bone. The hydrophilic bonds of the methylcellulose can cause the bone graft material to expand when water is absorbed. Combined with the collagen fibers, the methylcellulose can allow for the expansion of the bone graft material.

One of skill in the art will understand that methylcellulose in other iterations known in the art can cause problems related to terminal sterilization of the bone graft, such as causing crosslinking and poor performance post-sterilization. This can occur with gamma radiation, electron beam, and/or other sterilization methods. Methylcellulose that is crosslinked can result in poor handling. Methylcellulose of a lower molecular weight can improve handleability before sterilization, while methylcellulose of a higher molecular weight can improve handleability after terminal sterilization.

In certain examples, the methylcellulose can have a molecular weight between approximately 200 and approximately 1000. In some embodiments, the methylcellulose can have a molecular weight between approximately 100 and approximately 1500. For improved handleability before sterilization, for example, the methylcellulose can have a molecular weight of approximately 250. In some embodiments, for improved handleability before sterilization, for example, the methylcellulose can have a molecular weight between approximately 100 and 400. For improved handleability after sterilization, for example, the methylcellulose can have a molecular weight of approximately 700. In some embodiments, for improved handleability after sterilization, for example, the methylcellulose can have a molecular weight between approximately 650 and 750. In some embodiments, for improved handleability after sterilization, for example, the methylcellulose can have a molecular weight between approximately 500 and 1000. In some embodiments, for improved handleability after sterilization, for example, the methylcellulose can have a molecular weight between approximately 400 and 1500.

In some examples, using methylcellulose with a higher molecular weight, for example greater than 500, can be advantageous for this bone graft material. In some embodiments, the values for molecular weight can be in terms of g/mol. The bone graft material is sterilized once placed in the same vial, for example using gamma radiation or e-beam. The methylcellulose with a higher molecular weight can reduce the chances of being destroyed, denatured, degraded, or cross-linked by radiation. Bone graft materials in the field often use methylcellulose with a molecular weight between approximately 100 and 300, which can cause problems upon sterilizing the methylcellulose. Advantageously, using methylcellulose with a higher molecular weight can allow for the use of sterilized methylcellulose in the bone graft material without compromising the effectiveness of the methylcellulose.

In certain examples, a small amount of methylcellulose can be used, such as the lowest amount to optimize handling and bone growth. A large amount of methylcellulose may impede bone growth and prevent fusion.

Certain examples of bone graft material described herein may include Bioglass. For example, the bone graft material can include calcium sodium phosphosilicate, or 45S5 Bioglass. Bioglass can assist with bone growth. Bioglass can act as a scaffold, or a temporary template, for bone regeneration and actively stimulate vascularized bone growth. In some embodiments, calcium sodium phosphosilicate can be used.

In certain examples, the minimal amount of Bioglass needed to create a quick inflammatory response to recruit cells for healing and then resorb quickly to prevent sustained inflammation can be used. One of skill in the art will understand that exceedingly large particles may resorb too quickly and not provide long-term scaffolding needed to obtain fusion. Further, exceedingly small particles may cause long term inflammation and scar tissue formation. Without the collagen and carbonate apatite, the small Bioglass particles may result with inadequate bone formation due to impacting the rate of resorption. In some examples, the bone graft material can exclude Bioglass. Bioglass particle size can vary since a small amount is utilized. Sizes ranging from 1 micron to 25 microns may be used or 5 to 15 microns. An average of 10 microns can be a good average to stimulate bone growth. The small Bioglass particles can be in a powder form which allows it to help act as a carrier when fluid is added. When combined with carbonate apatite, methylcellulose can assist in handling and cohesiveness.

In some examples, the bone graft material described herein can include collagen. For example, the bone graft material can include fibrillar collagen, such as non-sterile, fibrillar collagen. Other suitable types of collagen can be used in this bone graft formulation as needed. In particular examples, the collagen is not lyophilized, freeze-dried, or formed into a collagen sponge. The collagen can have long fibers that can interlace the particles of the bone graft material and prevent migration. Collagen can also improve handling. Using fibrillar collagen can reduce the amount of time needed to mix with the methylcellulose and Bioglass. Collagen can also act as a carrier to help vascularize the area between the particles and hold stem cells, BMA, etc. In some implementations, the collagen can be loose collagen. For example, a kit can include loose, dry, fibers of collagen. The loose, dry, fibers of collagen can be non-lyophilized. In some implementations, a kit can include collagen that is unbound from the carbonate apatite, methylcellulose, and Bioglass. The kit can include fibrillar collagen with a length of greater than 5 mm. For example, the kit can include fibrillar collagen with a length of between 5 mm and 10 mm. In some embodiments, the kit can include fibrillar collagen with a length of between 5 mm and 20 mm. In some embodiments, the kit can include fibrillar collagen with a length of between 5 mm and 50 mm.

In examples, the collagen can be highly condensed and later separated. Highly condensed collagen can be separated, and the collagen can be fluffed up, for example using radiation. The fluffed up collagen can be cut. Then, the collagen can be added to the bone graft material. Collagen may be lyophilized to allow for optimal particle binding.

The carbonate apatite, methylcellulose, Bioglass, and collagen can be mixed homogeneously and then lyophilized to form a sponge, or a more rigid substance. Advantageously, a lyophilized bone graft material can be placed in the lateral gutters of a surgery, such as in spine surgery or other suitable surgeries.

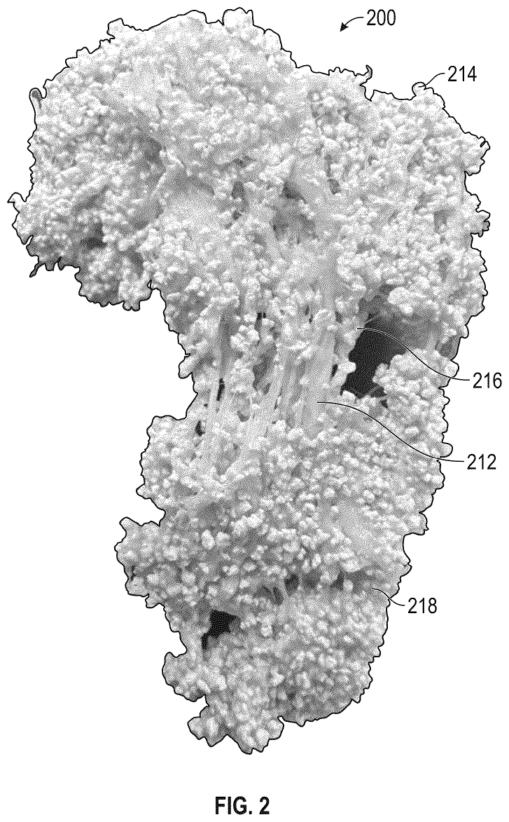

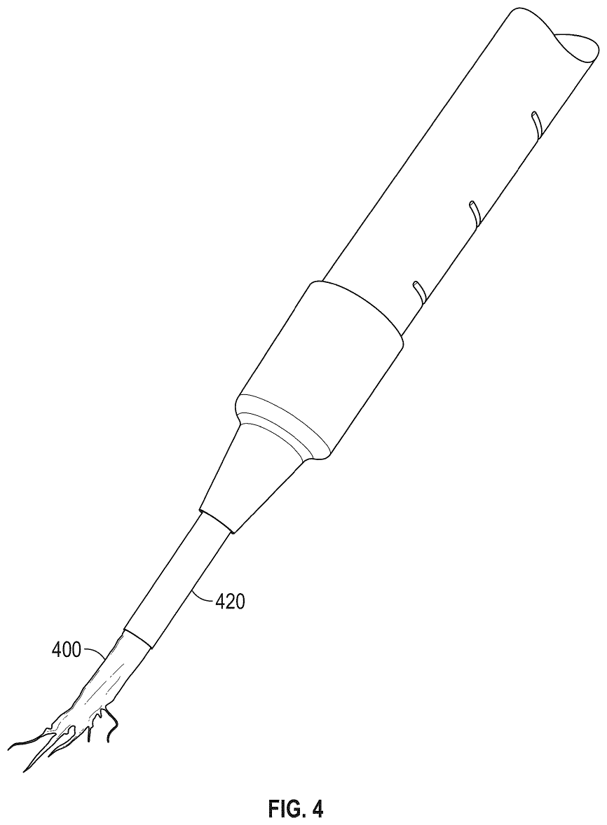

In certain examples, the bone graft material may include: carbonate apatite, Bioglass, methylcellulose, and collagen, which can form into a material mixture that may be optimally cohesive and handleable. The material can hold together seamlessly, as described with respect to FIGS. 2, 3F, 6A, 6B, 7A, and 7B. The material can flow through small apertures, as described with respect to FIG. 4. When placed in an aqueous solution, the particles can avoid migration. Advantageously, this formulation can minimize the use of methylcellulose while preventing particle migration.

As shown at block 104, in certain examples, the bone graft material can be sterilized using gamma radiation, electron beam, and/or another sterilization method. After being sterilized, the materials can be transported, for example to the site of a surgery. In some embodiments, sterilization can occur through pasteurization, or heating the bone graft material. In some embodiments, the bone graft material can be sterilized without cross-linking due to the properties of the collagen, methylcellulose, and Bioglass. In a preferred embodiment, the form of sterilization causes enough heat to change the structure of the collagen. For example, gamma radiation can be used.

As shown at block 106, in examples, the bone graft material can be mixed with an aqueous solution. In some embodiments, the aqueous solution can include blood, saline, bone marrow aspirate, stem cells, and/or another liquid for rehydration. The aqueous solution can contain growth factors to enhance bone healing. Rehydration can cause the bone graft material to turn from a dry material to a putty formulation. The putty formulation can be an amorphous, elastic, and/or malleable solid. The putty formulation can be cohesive with improved flowability. Advantageously, the cohesiveness of the bone graft material in the putty formulation can improve the ability of the bone graft to heal and grow bone and prevent non-unions. Because the bone graft is amorphous, it can fit into small apertures while being injected into a patient's body.

As shown at block 108, the bone graft material can be injected to a target site. The target site can be within a patient's body in contact with a bone. The bone graft material can be injected using a bone graft delivery system. For example, a rasp can be inserted into an incision and bone graft material can be delivered using the rasp.

In certain examples, the bone grafts described herein can be used in medical procedures including posterior lateral fusion procedures, sacroiliac joint fusion procedures, subtalar fusion procedures, hip revision procedures, knee revision procedures, Charcot joint procedures, and dental surgery. The bone grafts can be inserted into any incision suitable for reaching a desired surgical location, such as a facet joint, transverse process, disc space or sacroiliac joint, hip, ankle, tibia etc. The bone graft material can be used to fill a spinal, orthopedic, or dental implant, such as a cage or a screw.

In some examples, a container, vial, or kit can include calcium phosphate, for example carbonate apatite or calcium carbonate, Bioglass, methylcellulose, and collagen such as described herein. The carbonate apatite, Bioglass, methylcellulose, and collagen can be mixed homogenously. The carbonate apatite, Bioglass, methylcellulose, and collagen can be reconstituted in an aqueous solution.

FIG. 2 illustrates an example of bone graft material 200. As described with respect to FIG. 1, the bone graft material 200 can include collagen 212, carbonate apatite 214, methylcellulose 216, and Bioglass 218.

In examples, the collagen 212 can be fibrillar collagen that is not lyophilized. The carbonate apatite 214 can be pellets, spheres, or granules of carbonate apatite. The carbonate apatite 214 can be embedded in the collagen 212 when mixed with an aqueous solution. The methylcellulose 216 can be a powder. The methylcellulose 216 can be embedded in the collagen 212 when mixed with an aqueous solution. The Bioglass 218 can be a powder. The Bioglass 218 can be embedded in the collagen 212 when mixed with an aqueous solution. The bone graft solution 200 can have the consistency of a putty or paste. The bone graft solution 200 can be malleable, handleable, and cohesive. In some embodiments, the fibrillar collagen can be lyophilized. The bone graft material 200 can be cohesive and flowable but is not necessarily homogeneous. The bone graft material 200 can be biphasic, triphasic, quadphasic such as bioglass, a-tcp, b-tcp, or hydroxyapatite. The bone graft material 200 can be inserted into a body of a patient without a carrier, for example a porcine gel.

In certain examples, the bone graft material 200 can be porous based on the carbonate apatite 214. Carbonate apatite 214 can have micro porosity and macro porosity. Micro porosity within the particles themselves can allow for revascularization and bone growth. Macro porosity between the particles can create areas of revascularization and bone growth. Micro porosity and macro porosity can be important in to regenerate bone adequately and efficiently. The carbonate apatite can be developed through a process of sintering, or controlling the synthesis conditions. The carbonate apatite can be sintered such that the material has enhanced integration and osteoconductivity. This can also improve carbonate apatite's mechanical strength and stability. Advantageously, the carbonate apatite can maintain the structural integrity while being under pressure and weight during surgery and after surgery. This can be important to maintain a sufficient scaffold for growing bone.

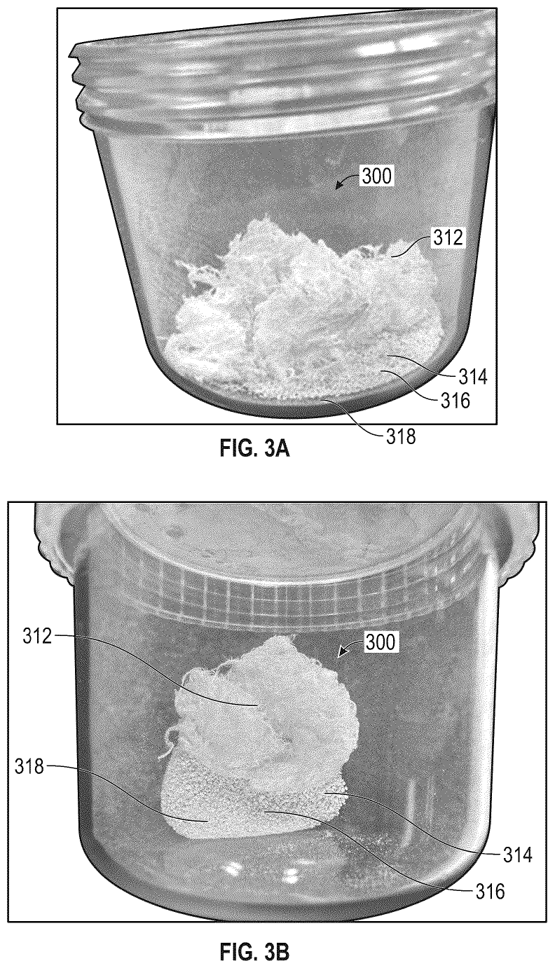

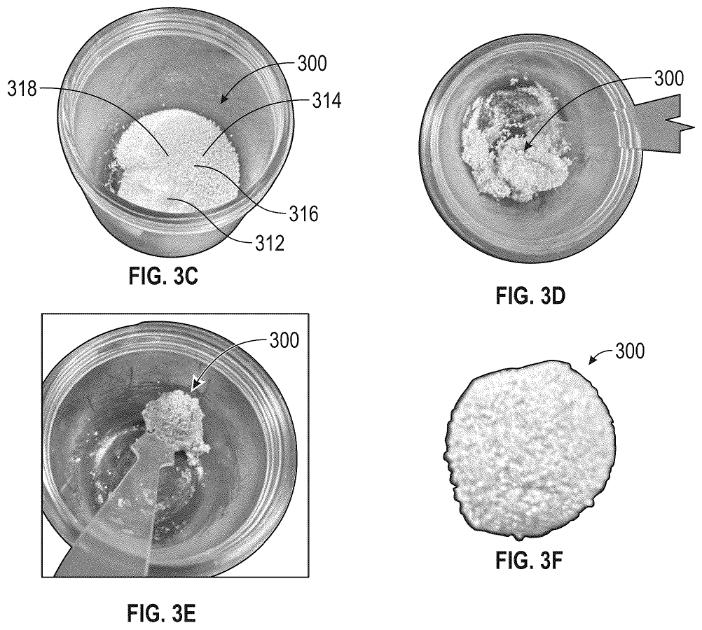

FIG. 3A illustrates an example of bone graft material 300 before sterilization. FIG. 3B illustrates an example of the bone graft material 300 of FIG. 3A after sterilization. FIG. 3C illustrates another example of the bone graft material 300 of FIG. 3A after sterilization. FIG. 3D illustrates an example of the bone graft material 300 of FIG. 3A after sterilization and during mixing with aqueous solution. FIG. 3E illustrates an example of the bone graft material 300 of FIG. 3A after sterilization and after mixing with aqueous solution. FIG. 3F illustrates an example of the bone graft material 300 of FIG. 3A after being sterilized, mixed with aqueous solution, and formed into a ball.

As shown in FIG. 3A, in some examples, the collagen 312 can be thin fibers of fibrillar collagen. The collagen 312 can be cut before being placed in a vial. The collagen 312 can be placed in a vial with carbonate apatite 314, methylcellulose 316, and Bioglass 218. As shown in FIG. 3B, when the bone graft material 300 is exposed to radiation, for example gamma radiation or e-beam radiation for sterilization, the collagen 312 can increase in volume. The structure of the collagen 312 can become fuller after sterilization.

As shown in FIG. 3C, 3D, and 3E, the collagen 312, carbonate apatite 314, methylcellulose 316, and Bioglass 318 can be mixed with an aqueous solution to allow the methylcellulose and carbonate apatite 314 to embed in the collagen 312. As shown in FIG. 3F, the result is a malleable bone graft material 300 that can be formed into a ball. Advantageously, the malleable bone graft material 300 can be formed to different shapes and has an enhanced ability to fill a target area without significant deforming or migrating.

FIG. 4 illustrates an example of bone graft material 400 being pushed through a nozzle 420 of a device. The bone graft material 400 can be similar to the bone graft material 200, 300 of FIGS. 2 and 3A-3F. The bone graft material 400 can be malleable such that it can fit through a nozzle 420 of a device. Advantageously, this can allow the bone graft material 400 to be injected into a patient or positioned near a target site.

The bone graft material 400 can cohesively flow through the device and out the nozzle 420, or the distal end of the device. Flowing cohesively can mean flowing continuously and in an unbroken, uniform stream. Flowing cohesively can mean the bone graft material is a viscoelastic material, such as a putty or a paste, rather than a powder or liquid that does not remain cohesive. The bone graft material 400 can be viscous and elastic, allowing it to deform under applied stress and subsequently remain in a certain structure. The bone graft material 400 can adapt to the contours of its surroundings.

In some examples, the nozzle 420 can be 2 mm in diameter. The nozzle 420 may be 1-5 mm in diameter. In some embodiments, the nozzle 420 can be 0.5-10 mm in diameter.

In certain examples, the bone graft material 400 can be delivered using a bone graft delivery device. The bone graft delivery device can include a handle having a trigger or other actuation mechanism, a tube having a lumen therethrough, and a distal tip. The bone graft delivery device can be similar to a caulking gun. The handle can house a supply of the desired bone graft material. The bone graft material 400 can be pre-loaded in the handle or tube or can be supplied to the handle, for example, via a cartridge that can be removably coupled to the handle. In some embodiments, the device can further include a plunger that is retracted proximally to allow the handle to receive a cartridge or pre-loaded volume of bone graft material.

In use, the trigger can be actuated to deliver bone graft material 400 through the tube and distal tip or nozzle 420 to a desired surgical location. In some embodiments, the plunger is simultaneously pushed distally to help deliver bone graft material through the tube. In some embodiments, the trigger or other actuation mechanism is configured to deliver a controlled release amount of bone graft material during actuation of the device, for example, ½ cc of bone graft material per complete squeeze of the trigger. The trigger or other actuation mechanism may be operated manually or by mechanical, battery powered, electric, pneumatic, or any other means of force.

In some embodiments, a portion of the handle can include an opening configured to receive the bone graft material. For example, a base of the handle can include a funnel. In other embodiments, a side or another portion of the handle can include a funnel or other opening configured to receive the bone graft material.

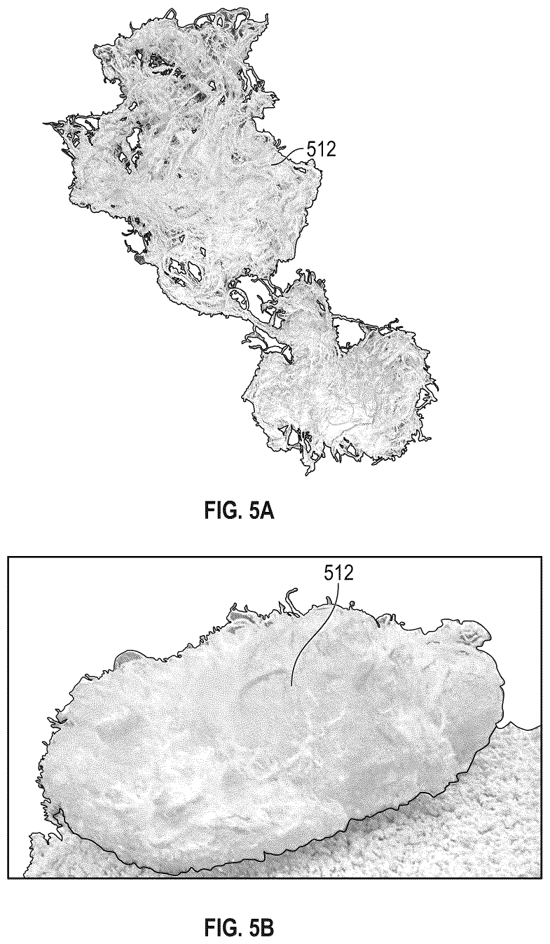

FIG. 5A illustrates an example of collagen 512 before sterilization. FIG. 5B illustrates an example of the collagen 512 of FIG. 5A after sterilization. As described with respect to FIGS. 3A and 3B, the collagen can increase in volume or fluff up when exposed to radiation. The fluffed up collagen can advantageously wick up more bone marrow aspirate, or other aqueous solution, than the collagen before sterilization.

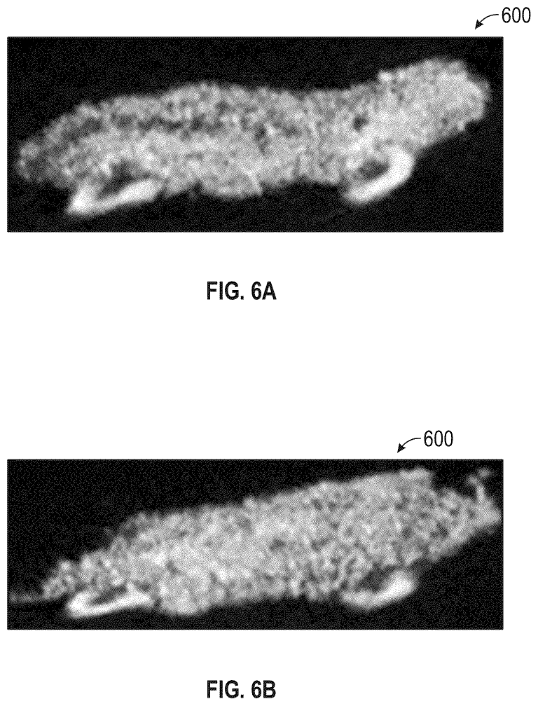

FIGS. 6A-B illustrate an example of a sagittal cut CT scan of the bone graft material 600 after posterolateral fusion. The bone graft material 600 is shown from a sagittal cut CT scan from a posterolateral fusion rabbit study. The bone graft material 600 can be similar to the bone graft material 200, 300, 400 of FIGS. 2, 3A-F, and 4. The bone graft material 600 can revascularize a target area and induce healing. Because of the fibrillar collagen, the bone graft material 600 can be cohesive and avoid migration in the body.

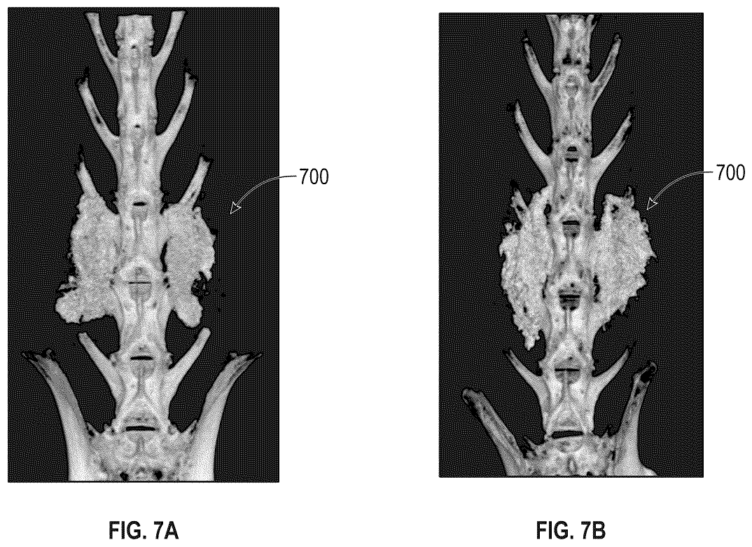

FIGS. 7A-B illustrate an example of a coronal view CT scan of the bone graft material 700 after posterolateral fusion. The bone graft material 700 is shown from a coronal view CT scan from a posterolateral fusion rabbit study. The bone graft material 700 can be similar to the bone graft material 200, 300, 400 of FIGS. 2, 3A-F, and 4. The bone graft material 700 requires enough structure to reach certain parts of the target site once placed in the body. The collagen can provide enough structure such that the methylcellulose and carbonate apatite can reach the necessary areas.

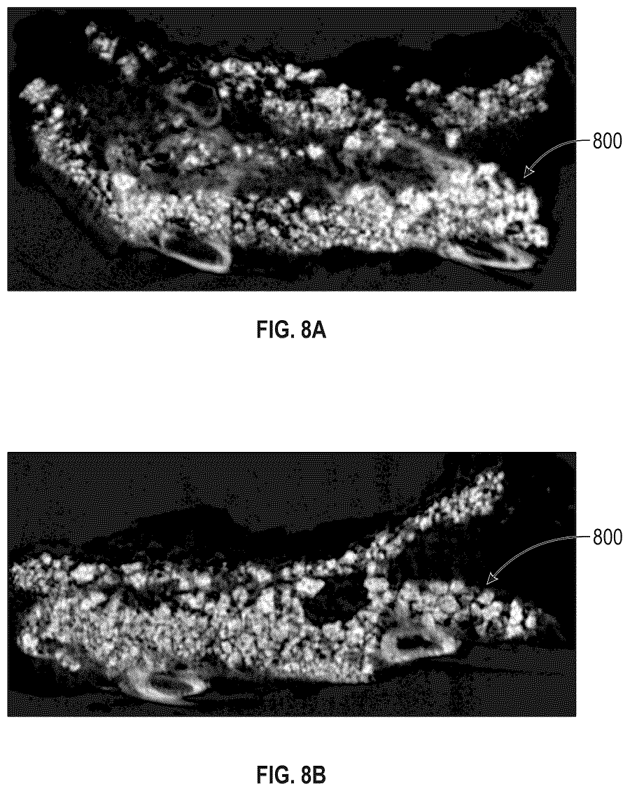

FIGS. 8A-B illustrate an example of a CT scan of the bone graft material 800 without collagen after posterolateral fusion. The bone graft material 800 is shown from a CT scan from a bone void study. The bone graft material 800 can be similar to the bone graft material 200, 300, 400 of FIGS. 2, 3A-F, and 4, but without collagen. Because of the bone void obviating the need for cohesiveness by preventing the particles from migrating, the bone graft material 800 can be effective without collagen. The bone graft material 800 can be contained within the void to prevent migration. The bone graft material 800 can disperse to some extent because of the lack of collagen.

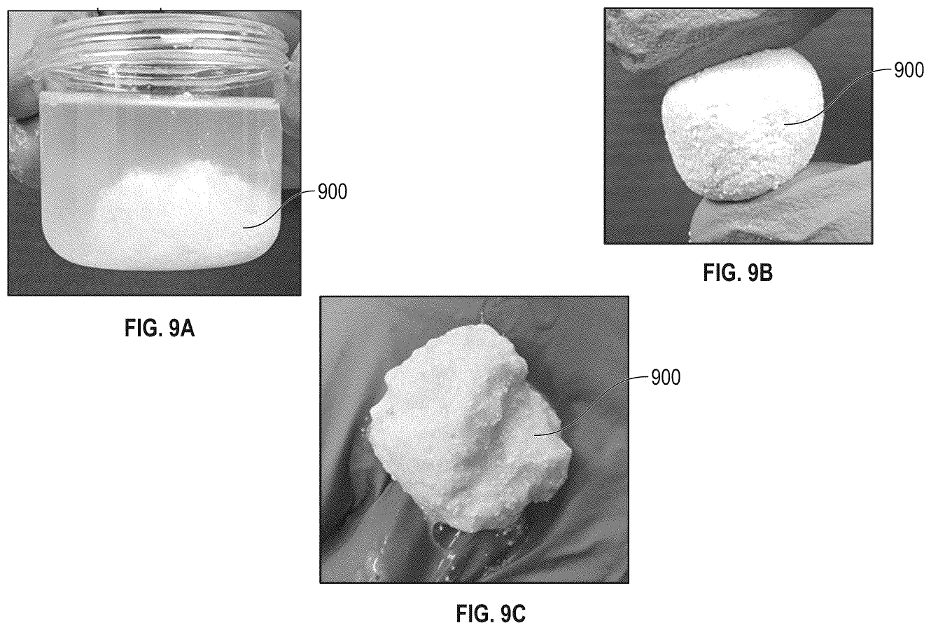

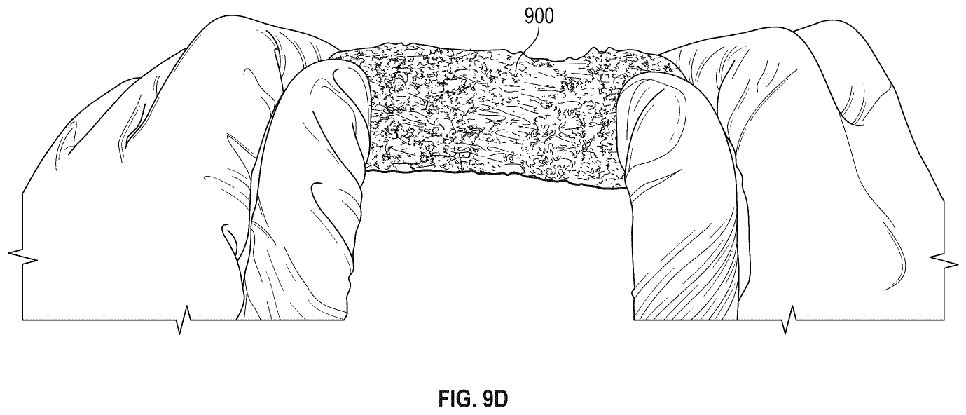

FIG. 9A shows an example of bone graft material 900 in an aqueous solution. FIG. 9B shows the example of the bone graft material 900 of FIG. 9A compacted into a ball. FIG. 9C shows the example of the bone graft material 900 of FIG. 9A in an expanded state. FIG. 9D shows the example of the bone graft material 900 of FIG. 9A being pulled in different directions.

The bone graft material 900 can be similar to the bone graft material described with respect to FIGS. 1-8B herein.

The bone graft material 900 can include loose collagen fibers. The collagen fibers can be unbound. The collagen fibers can be not pre-mixed. The collagen fibers can be not lyophilized.

The bone graft material 900 can include methylcellulose. The methylcellulose can help bond particles to the collagen fibers. In some examples, without methylcellulose, the particles can have difficulty mixing properly. With the methylcellulose, the bone graft material 900 can stay bonded or cohesive. Advantageously, the methylcellulose can allow for integration of the elements of the bone graft material 900. The methylcellulose can therefore allow for the viscoelastic properties of the bone graft material 900. The methylcellulose can allow the particles to remain in the target site near the bone rather than migrating. The methylcellulose can be hydrophilic. The methylcellulose and collagen can wick up bone marrow aspirate. The methylcellulose can have a viscoelastic, or jelly-like, consistency. Advantageously, this can allow the bone graft material 900 to adhere to itself and surrounding structures.

The bone graft material 900 may be homogeneous. The bone graft material 900 may be fibrous. The bone graft material 900 may be moldable. The bone graft material 900 may be cohesive. The bone graft material 900 may not fall apart or migrate.

As shown in FIGS. 9A and 9B, the bone graft material 900 can expand over time. FIG. 9B shows the bone graft material 900 compacted, and FIG. 9C shows the bone graft material 900 after some expansion. In FIG. 9C, the bone graft material can 900 be shown after being in an aqueous solution for approximately 48 hours. Advantageously, the bone graft material 900 can remain cohesive after being in the aqueous solution for less than or equal to 48 hours. Advantageously, the expansion of the bone graft material 900 can allow the bone graft material 900 to fill in a void. A user may implant the bone graft material 900 in a tumor void, a cage, a bone void, a gutter, and/or an interface between bones. Over time, the bone graft material 900 can expand to push up against the margins of the space. Advantageously, this can promote bone fusion at an interface between bones. Other bone graft materials often break down over time, causing their effectiveness to reduce over time. This occurs because other bone graft materials have particles and a carrier. Once the carrier resorbs, the bone graft drops because the volume reduces. This reduces the effectiveness of initiating fusion over time. Because this bone graft material 900 expands over time, its effectiveness can be maintained or increase over time.

The bone graft material 900 can expand due to additional hydration. The collagen fibers may be at least partially dehydrated and/or have capacity for additional hydration. The combination of the collagen fibers and the carboxymethylcellulose can allow the bone graft material 900 can take on additional hydration and expand as a result. Conversely, a collagen sponge, or lyophilized collagen, would not expand sufficiently. Additionally, the collagen sponge is not sufficiently moldable to fit through a nozzle of a device. The collagen of the bone graft material 900 is processed in bands such that the collagen is malleable and expandable.

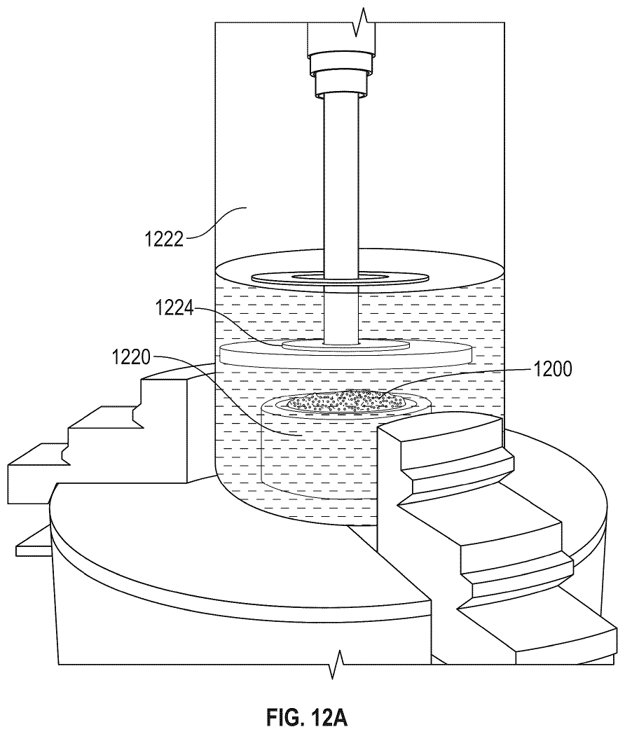

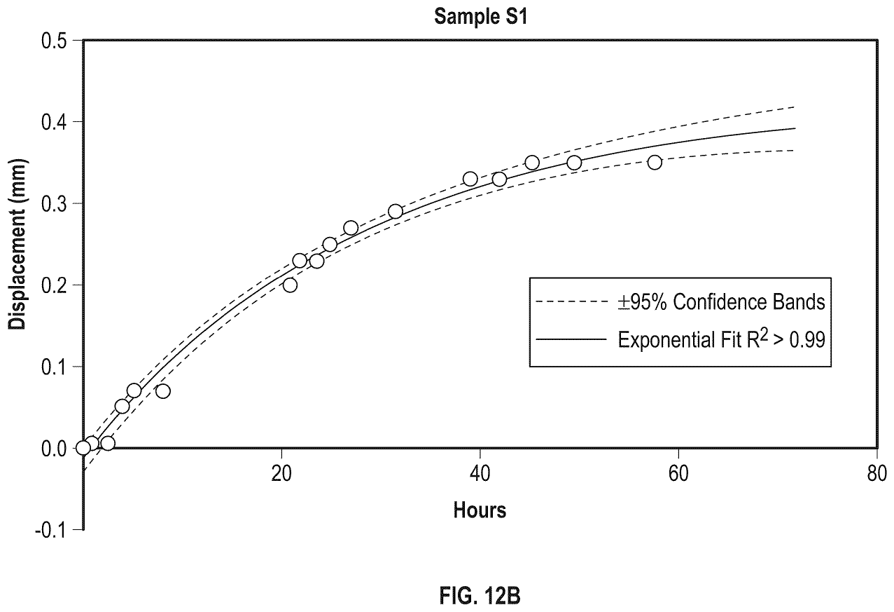

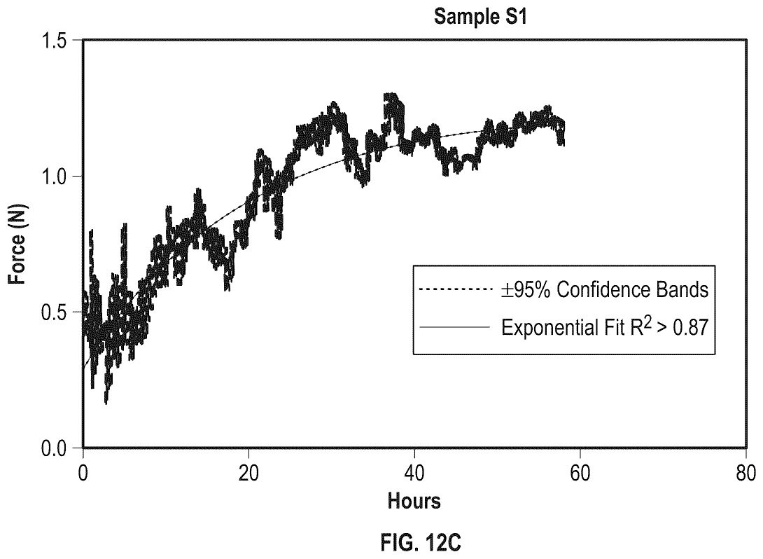

Osteons have a finite distance for initiation of bone growth. Beyond this critical distance, growth may be delayed or absent. The critical distance which osteons may initiate bone growth is approximately 0.024 mm. Advantageously, a bone graft material that can expand at least 0.024 mm can allow for the initiation of bone growth. As shown with respect to FIG. 12A, an embodiment of the bone graft material 900 can expand greater than 0.35 mm. In some embodiments, the bone graft material 900 can expand between approximately 0.35 mm and 0.5 mm. In some embodiments, the bone graft material 900 can expand between approximately 0.1 mm and 0.75 mm. In some embodiments, the bone graft material 900 can expand between approximately 0.05 mm and 1 mm. In some embodiments, the bone graft material 900 can expand between approximately 0.025 mm and 1.5 mm. Existing bone graft materials do not expand at least 0.024 mm after being implanted. Rather, existing bone graft materials decrease in size after implantation.

The bone graft material 900 may be reconstituted in bone marrow aspirate. Advantageously, this can bond the bone marrow aspirate with the methylcellulose. This can allow live cells and growth factors for growing bone to be bonded with the bone graft material 900.

Many existing bone graft materials, for example with lyophilized collagen, are cut and placed in a void or target site. Because cutting is imprecise, many existing bone graft materials do not precisely fill the void or target site. Because the bone graft material 900 expands, it can completely fill the void or target site.

As shown in FIG. 9D, applying force in opposite directions to the bone graft material 900 can cause the bone graft material 900 to stretch. The collagen fibers of the bone graft material 900 can cause the bone graft material 900 to remain cohesive while being pulled in different directions. Advantageously, the collagen fibers can maintain cohesiveness while allowing the bone graft material 900 to expand in the target site of the patient and/or move through devices during insertion.

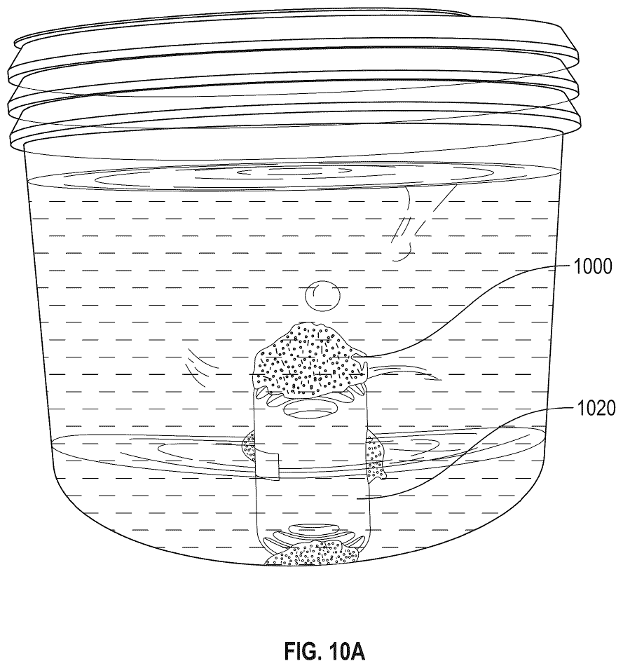

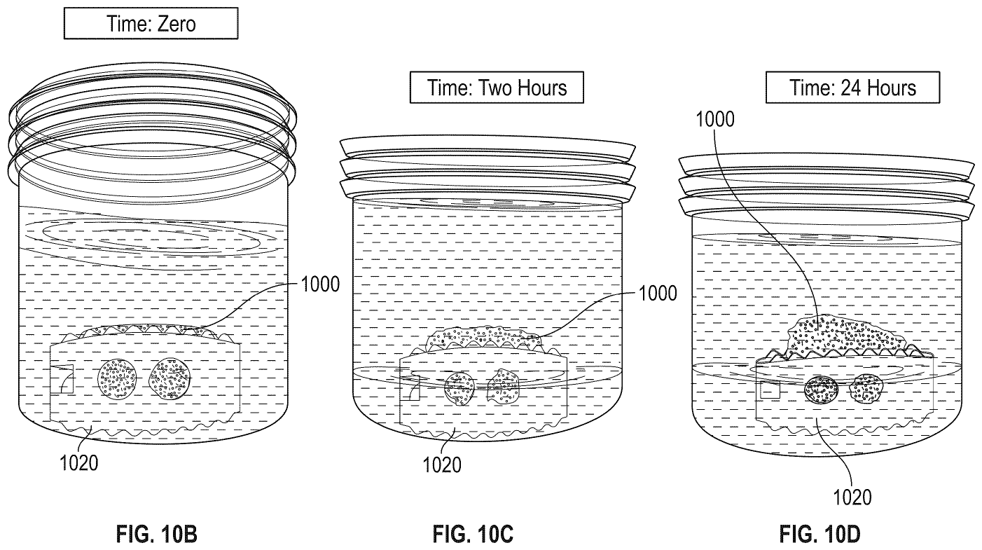

FIG. 10A shows an example of bone graft material 1000 in an posterior lumbar interbody fusion (PLIF) cage 1020. FIG. 10B shows the example of bone graft material 1000 in the PLIF cage 1020 of FIG. 10A at a first time. FIG. 10C shows the example of bone graft material 1000 in the PLIF cage 1020 of FIG. 10A at a second time. FIG. 10D shows the example of bone graft material 1000 in the PLIF cage 1020 of FIG. 10A at a third time.

The bone graft material 1000 can be similar to the bone graft material described with respect to FIGS. 1-9D herein.

The bone graft material 1000 can be packed into an PLIF cage 1020. An PLIF cage 1020 can be implanted between two vertebrae in the lower back. In some embodiments, the PLIF cage 1020 can be implanted in an area where arthritis or a tumor was removed, leaving a void. The PLIF cage 1020 may not contact the surrounding bone sufficiently due to the asymmetry of the patient's bone and variances in anatomy. Bridging bone is important for the long-term outcome of the patient's spine. Advantageously, the expandable bone graft material 1000 allows the entire region to heal.

As shown with respect to FIGS. 10B, 10C, and 10D, the bone graft material 1000 may expand from the top and/or bottom of the PLIF cage 1020. Advantageously, the bone graft material 1000 may fill space in the void around the PLIF cage 1020. The PLIF cage 1020 does not necessarily fit perfectly in the target area, so the bone graft material 1000 can help fill the target area. As shown in FIG. 10B, the bone graft material 1000 can be packed into the PLIF cage 1020. As shown in FIG. 10C, the bone graft material 1000 can expand from the top and/or bottom of the PLIF cage after two hours. As shown in FIG. 10D, the bone graft material 1000 can further expand from the top and/or bottom of the PLIF cage after 24 hours.

The PLIF cage 1020 can replace a vertebral disc. The PLIF cage 1020 can be placed between vertebrae. The PLIF cage 1020 can be placed in the back of the spine. The bone graft material 1000 can expand to contact the vertebrae and initiate bone growth. The bone can grow around the PLIF cage 1020 such that the vertebrae is repaired. The PLIF cage 1020 can provide structural integrity in the vertebrae while the bone graft material 1000 heals the bone and/or fills the void.

The bone graft material 1000 can rehydrate in the patient's body such that it expands from the top and/or bottom of the PLIF cage 1020. In some embodiments, the bone graft material 1000 can expand between approximately 0.35 mm and 0.5 mm from the top and/or bottom of the PLIF cage 1020. In some embodiments, the bone graft material 1000 can expand between approximately 0.1 mm and 0.75 mm from the top and/or bottom of the PLIF cage 1020. In some embodiments, the bone graft material 1000 can expand between approximately 0.05 mm and 1 mm from the top and/or bottom of the PLIF cage 1020. In some embodiments, the bone graft material 1000 can expand between approximately 0.025 mm and 1.5 mm from the top and/or bottom of the PLIF cage 1020. In some embodiments, the bone graft material 1000 can expand between approximately 0.001 mm and 5 mm from the top and/or bottom of the PLIF cage 1020. In some embodiments, the bone graft material 1000 can expand from the top and/or bottom of the PLIF cage 1020 a sufficient amount to reach the vertebrae. In some embodiments, the bone graft material 1000 can expand from the top and/or bottom of the PLIF cage 1020 a sufficient distance to fill the void area.

A user can pack the PLIF cage 1020 with bone graft material 1000. The bone graft material 1000 can take the shape of the interior of the PLIF cage 1020 while retaining its structure due to its viscoelastic properties. The user can perform a discectomy, or remove a damaged intervertebral disc. The user can position the PLIF cage 1020 in the void where the intervertebral disc was removed. The bone graft material 1000 can hydrate in the body to expand to contact adjacent vertebrae. For example, the bone graft material 1000 can hydrate due to contact with blood and/or cerebrospinal fluid. The bone graft material 1000 can wick up bone marrow aspirate in the body. The bone graft material 1000 can promote fusion of adjacent vertebrae over time.

Other cages may be packed with the bone graft material 1000. The bone graft material 1000 may be packed into an expandable cage. An expandable cage allows swelling and bulging. When the bone graft material 1000 expands, the expandable cage would also expand to allow the two surrounding vertebrae to be fused and connected.

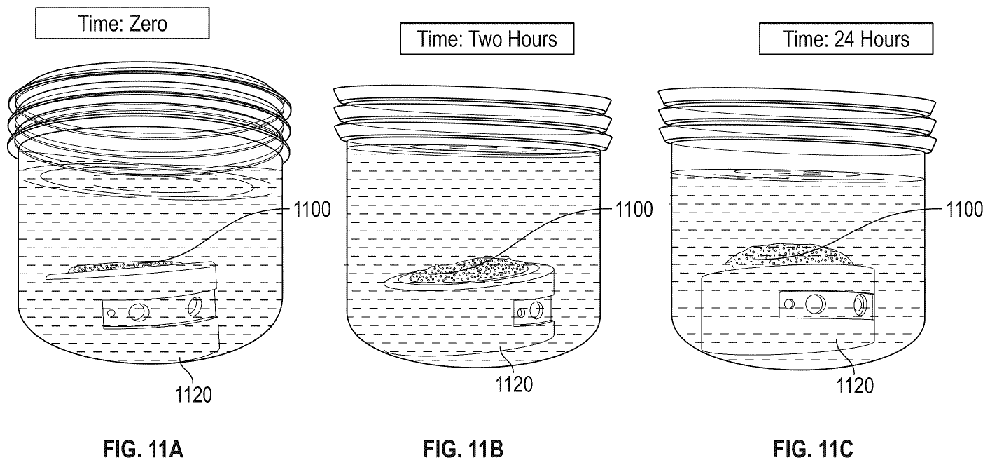

FIG. 11A shows an example of bone graft material 1100 in an anterior lumbar interbody fusion (ALIF) cage 1120 at a first time. FIG. 11B shows the example of bone graft material 1100 in the ALIF cage 1120 of FIG. 11A at a second time. FIG. 11C shows the example of bone graft material 1100 in the ALIF cage 1120 of FIG. 11A at a third time.

The bone graft material 1100 can be similar to the bone graft material described with respect to FIGS. 1-10D herein.

The bone graft material 1100 can be packed into an ALIF cage 1120. An ALIF cage 1120 can be implanted between two vertebrae in the lower back. In some embodiments, the ALIF cage 1120 can be implanted in an area where arthritis or a tumor was removed, leaving a void. The ALIF cage 1120 may not contact the surrounding bone sufficiently due to the asymmetry of the patient's bone and variances in anatomy. Bridging bone is important for the long-term outcome of the patient's spine. Advantageously, the expandable bone graft material 1100 allows the entire region to heal.

As shown with respect to FIGS. 11A, 11B, and 11C, the bone graft material 1100 may expand from the top and/or bottom of the ALIF cage 1120. Advantageously, the bone graft material 1100 may fill space in the void around the ALIF cage 1120. The ALIF cage 1120 does not necessarily fit perfectly in the target area, so the bone graft material 1100 can help fill the target area. As shown in FIG. 11A, the bone graft material 1100 can be packed into the ALIF cage 1120. As shown in FIG. 11B, the bone graft material 1100 can expand from the top and/or bottom of the ALIF cage after two hours. As shown in FIG. 11C, the bone graft material 1100 can further expand from the top and/or bottom of the ALIF cage after 24 hours.

The ALIF cage 1120 can replace a vertebral disc. The ALIF cage 1120 can be placed between vertebrae. The ALIF cage 1120 can be placed in the front of the spine. The bone graft material 1100 can expand to contact the vertebrae and initiate bone growth. The bone can grow around the ALIF cage 1120 such that the vertebrae is repaired. The ALIF cage 1120 can provide structural integrity in the vertebrae while the bone graft material 1100 heals the bone and/or fills the void.

The bone graft material 1100 can rehydrate in the patient's body such that it expands from the top and/or bottom of the ALIF cage 1120. In some embodiments, the bone graft material 1100 can expand between approximately 0.35 mm and 0.5 mm from the top and/or bottom of the ALIF cage 1120. In some embodiments, the bone graft material 1100 can expand between approximately 0.1 mm and 0.75 mm from the top and/or bottom of the ALIF cage 1120. In some embodiments, the bone graft material 1100 can expand between approximately 0.05 mm and 1 mm from the top and/or bottom of the ALIF cage 1120. In some embodiments, the bone graft material 1100 can expand between approximately 0.025 mm and 1.5 mm from the top and/or bottom of the ALIF cage 1120. In some embodiments, the bone graft material 1100 can expand between approximately 0.001 mm and 5 mm from the top and/or bottom of the ALIF cage 1120. In some embodiments, the bone graft material 1100 can expand from the top and/or bottom of the ALIF cage 1120 a sufficient amount to reach the vertebrae. In some embodiments, the bone graft material 1100 can expand from the top and/or bottom of the ALIF cage 1120 a sufficient distance to fill the void area.

A user can pack the ALIF cage 1120 with bone graft material 1100. The bone graft material 1100 can take the shape of the interior of the ALIF cage 1120 while retaining its structure due to its viscoelastic properties. The user can perform a discectomy, or remove a damaged intervertebral disc. The user can position the ALIF cage 1120 in the void where the intervertebral disc was removed. The bone graft material 1100 can hydrate in the body to expand to contact adjacent vertebrae. For example, the bone graft material 1100 can hydrate due to contact with blood and/or cerebrospinal fluid. The bone graft material 1100 can wick up bone marrow aspirate in the body. The bone graft material 1100 can promote fusion of adjacent vertebrae over time.

Other cages may be packed with the bone graft material 1100. The bone graft material 1100 may be packed into an expandable cage. An expandable cage allows swelling and bulging. When the bone graft material 1100 expands, the expandable cage would also expand to allow the two surrounding vertebrae to be fused and connected.

FIG. 12A shows an embodiment of bone graft material 1200 in an ALIF cage 1220 in a test apparatus. FIG. 12B is a graph of the displacement over time of an embodiment of the bone graft material described herein. FIG. 12C is a graph of the force exerted by an embodiment of the bone graft material described herein over time.

As shown in FIG. 12A, the bone graft material 1200 was packed into an ALIF cage 1220. The ALIF cage 1220 containing the material was placed in a container 1222 of liquid. A piston 1224 was positioned on top of the ALIF cage 1220. As the bone graft material expanded, pressure sensors on the piston 1224 were used to measure the force exerted by the bone graft material 1200. The distance the bone graft material 1200 expanded was also measured.

As shown in FIG. 12B, the bone graft material 1200 expanded over 0.05 mm within the first 10 hours. Advantageously, this is over twice the critical distance (0.024 mm) needed for bone growth. The bone graft material 1200 expanded approximately 0.2 mm after 20 hours. The bone graft material 1200 expanded approximately 0.35 mm after 50 hours. The bone graft material 1200 expanded approximately 0.4 mm after 70 hours. The expansion of the bone graft material 1200 was exponential, with expansion stabilizing at around 0.4 mm. The exponential fit of the force exerted was R²>0.99. Advantageously, the expansion of the bone graft material 1200 can allow a void to be filled without overexpanding or shrinking. In some examples, the bone graft material 1200 can expand less than or equal to 4 mm. In some examples, the bone graft material 1200 can expand less than or equal to 6 mm. Overexpansion could cause harm to surrounding structures. Shrinking could reduce the effectiveness of the graft over time.

As shown in FIG. 12C, the force exerted by the bone graft material 1200 on the piston 1224 was between approximately 0.5 N and 1.25 N between 10 hours and 60 hours. The force exerted by the bone graft material 1200 was exponential, with the force stabilizing at around 1.2 mm. The exponential fit of the force exerted was R² >0.87. Advantageously, this amount of force on the surrounding structures during implantation would not be harmful to the patient. The bone graft material 1200 can expand within a void without exerting more than 1.5 N on the surrounding structures.

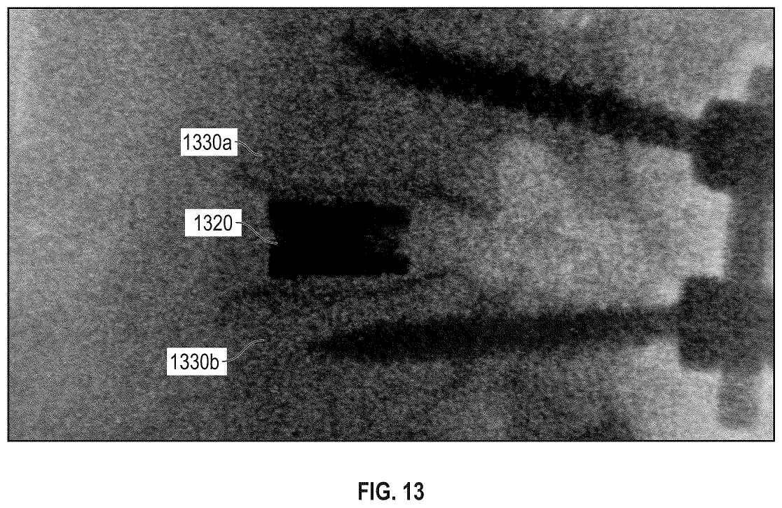

FIG. 13 shows an example of a cage 1320 positioned between vertebrae 1330a,b.

The space between the vertebrae and the cage 1320 may be filled with the expanding bone graft material described with respect to FIGS. 1-12C. As the bone graft material expands, it can contact the upper vertebrae 1330a and the lower vertebrae 1330b. This can promote fusion of the upper vertebrae 1330a and the lower vertebrae 1330b.

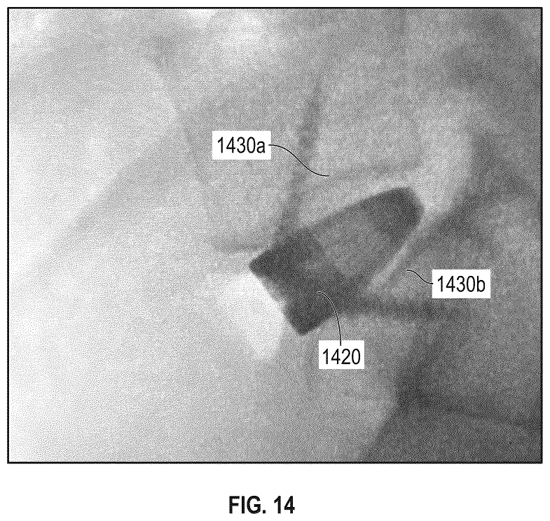

FIG. 14 shows another example of a cage 1420 positioned between vertebrae 1430a,b.

The space between the vertebrae and the cage 1420 may be filled with the expanding bone graft material described with respect to FIGS. 1-13. As the bone graft material expands, it can contact the upper vertebrae 1430a and the lower vertebrae 1430b. This can promote fusion of the upper vertebrae 1430a and the lower vertebrae 1430b. The lucent lines between the endplates of the vertebrae 1430a,b and the cage 1420 show space where the bone graft material can expand.

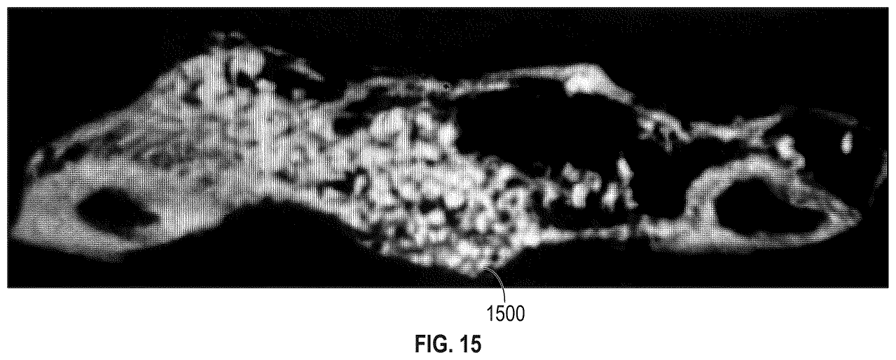

FIG. 15 shows a 3 month pre-clinical testing CT scan of a solid posterior lateral fusion in an animal model using an example of bone graft material 1500.

The example of the bone graft material 1500 can be the same as or similar to the bone graft material described with respect to FIGS. 1-14. The bone graft material 1500 may be used without the addition of an autograft, as shown in FIG. 15. Achieving fusion in a Boden model without an autograft can be accomplished due to the properties of the bone graft material 1500 described with respect to FIGS. 1-14. The ability of the bone graft material 1500 to expand can allow the material to function in cases that would normally require an autograft as a graft extender and/or graft enhancer. The bone graft material 1500 can function at least as well as an autograft for repairing bones or fusing joints.

Non-limiting Examples are included below.
Example 1. A method for forming a bone graft, the method comprising: mixing carbonate apatite, methylcellulose, Bioglass, and collagen to form a bone graft material, wherein the methylcellulose has a molecular weight of greater than 500; sterilizing the bone graft material; mixing the bone graft material with an aqueous solution; filling a device with the bone graft material; and inserting, using the device, the bone graft material into a patient such that it contacts a bone of the patient, wherein the bone graft material is viscoelastic, and wherein the bone graft material flows through a distal end of the device to contact the bone.
Example 2. The method of Example 1, wherein sterilizing the bone graft material comprises sterilizing the bone graft material using gamma radiation.
Example 3. The method of any one of Examples 1 and 2, wherein sterilizing the bone graft material comprises sterilizing the bone graft material using electron beam.
Example 4. The method of any one of Examples 1-3, wherein the aqueous solution comprises at least one of blood, saline, bone marrow aspirate, or stem cells.
Example 5. The method of any one of Examples 1-4, wherein the carbonate apatite has a particle size between 0.3 microns and 1 micron.
Example 6. The method of any one of Examples 1-4, wherein the carbonate apatite has a particle size between 0.3 microns and 2 microns.
Example 7. The method of any one of Examples 1-6, wherein the methylcellulose has a molecular weight of between 650 and 750.
Example 8. The method of any one of Examples 1-7, wherein the distal end of the device comprises a nozzle.
Example 9. The method of Example 8, wherein the nozzle has a diameter of 1-5 mm.
Example 10. The method of any one of Examples 1-9, wherein the collagen

is fibrillar collagen.
Example 11. The method of any one of Examples 1-10, wherein the collagen is not lyophilized.
Example 12. The method of claim any one of Examples 1-11, wherein inserting the bone graft material into the patient comprises: inserting a rasp into an incision; and delivering the bone graft material into the incision.
Example 13. A composition for a bone graft comprising: carbonate apatite; methylcellulose with a molecular weight of greater than 500; Bioglass; and fibrillar collagen, wherein the composition is viscoelastic, wherein the composition is configured to flow through a distal end of a device to a target area, and wherein the composition is configured to revascularize the target area.
Example 14. The composition of Example 13, wherein the carbonate apatite has a particle size between 0.3 microns and 1 micron.
Example 15. The composition of Example 13, wherein the carbonate apatite has a particle size between 0.3 microns and 2 microns.
Example 16. The composition of any one of Examples 13-15, wherein the methylcellulose has a molecular weight of between 650 and 750.
Example 17. The composition of any one of Examples 13-16, wherein the collagen is not lyophilized.
Example 18. The composition of any one of Examples 13-17, wherein the carbonate apatite, methylcellulose, and Bioglass are embedded in the collagen.
Example 19. A bone graft material comprising: fibrillar collagen; and methylcellulose, wherein the bone graft material expands at least 0.05 mm when hydrated.
Example 20. The bone graft material of Example 19, wherein the bone graft material exerts less than 1.5 N of force on surrounding structures during expansion.
Example 21. The bone graft material of Example 19, wherein the bone graft material expands at least 0.35 mm when hydrated.
Example 22. A method for promoting vertebrae fusion, the method comprising: packing a cage with bone graft material comprising fibrillar collagen and methylcellulose; removing an intervertebral disc from a patient to form a void; and positioning the cage in the void, wherein the bone graft material is configured to expand to contact an upper vertebrae and a lower vertebrae.
Example 23. The method of Example 22, wherein the cage is an anterior lumbar interbody fusion cage.
Example 24. The method of any one of Examples 22 or 23, wherein the cage is a posterior lumbar interbody fusion cage.
Example 25. The method of any one of Examples 22-24, wherein the bone graft material is at least partially dehydrated.
Example 26. The method of any one of Examples 22-25, wherein the bone graft material is configured to expand when hydrated.
Example 27. An apparatus substantially as shown and/or described.
Example 28. A method substantially as shown and/or described.
Example 29. A system substantially as shown and/or described.

The devices described herein can also be used in spinal procedures and other orthopedic applications to deliver bone graft material to other locations in the body (for example, the femur or tibia).

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the principles and features disclosed herein. Various combinations and subcombinations of the various features described herein are possible. Certain embodiments of the invention are encompassed in the claim set listed below.

Although this disclosure describes certain embodiments, it will be understood by those skilled in the art that many aspects of the methods and devices shown and described in the present disclosure may be differently combined and/or modified to form still further embodiments or acceptable examples. All such modifications and variations are intended to be included herein within the scope of this disclosure. Indeed, a wide variety of designs and approaches are possible and are within the scope of this disclosure. No feature, structure, or step disclosed herein is essential or indispensable. Moreover, while illustrative embodiments have been described herein, the scope of any and all embodiments having equivalent elements, modifications, omissions, combinations (e.g., of aspects across various embodiments), substitutions, adaptations and/or alterations as would be appreciated by those in the art based on the present disclosure. While certain embodiments have been described, these embodiments have been presented by way of example only, and are not intended to limit the scope of protection.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method for using a bone graft, the method comprising:
providing a bone graft material comprising calcium phosphate, methylcellulose, Bioglass, and collagen;
mixing the bone graft material with an aqueous solution; and
inserting the bone graft material into a patient such that it regenerates a bone of the patient.

2. The method of claim 1, wherein the methylcellulose has a molecular weight of greater than 500.

3. The method of claim 1, wherein the bone graft material is sterilized.

4. The method of claim 1, wherein the bone graft material is viscoelastic.

5. The method of claim 1, wherein the bone graft material flows through a distal end of a device to contact the bone.

6. The method of claim 1, wherein the calcium phosphate comprises carbonate apatite.

7. The method of claim 1, wherein the aqueous solution comprises at least one of blood, saline, bone marrow aspirate, or stem cells.

8. The method of claim 6, wherein the carbonate apatite has a particle size between 0.3 microns and 1 micron.

9. The method of claim 6, wherein the carbonate apatite has a particle size between 0.3 microns and 2 microns.

10. The method of claim 1, wherein the methylcellulose has a molecular weight of between 650 and 750.

11. The method of claim 5, wherein the bone graft material flows through a nozzle of the device, the nozzle having a diameter of 1-5 mm.

12. The method of claim 1, wherein inserting the bone graft material into the patient comprises:
inserting a rasp into an incision; and
delivering the bone graft material into the incision.

13. The method of claim 1, wherein the methylcellulose has a viscosity of between 5,000 cPs and 15,000 cPs.

14. A composition for a bone graft comprising:
calcium phosphate;
methylcellulose;
Bioglass; and
fibrillar collagen, wherein the fibrillar collagen is not lyophilized, and wherein the fibrillar collagen comprises loose fibers,
wherein the composition is configured to regenerate bone.

15. The composition of claim 14, wherein the calcium phosphate comprises carbonate apatite.

16. The composition of claim 14, wherein the methylcellulose has a molecular weight of greater than 500.

17. The composition of claim 14, wherein the composition is viscoelastic.

18. The composition of claim 14, wherein the composition is configured to flow through a distal end of a device to a target area.

19. The composition of claim 15, wherein the carbonate apatite has a particle size between 0.3 microns and 1 micron.

20. The composition of claim 15, wherein the carbonate apatite has a particle size between 0.3 microns and 2 microns.

21. The composition of claim 14, wherein the methylcellulose has a molecular weight of between 650 and 750.

22. The composition of claim 15, wherein the carbonate apatite, methylcellulose, and Bioglass are embedded in the fibrillar collagen.

23. The composition of claim 14, wherein the composition expands at least 0.05 mm when hydrated.

24. The composition of claim 14, wherein the composition exerts less than 1.5 N of force on surrounding structures during expansion.

25. The composition of claim 14, wherein the composition expands at least 0.35 mm when hydrated.

26. The composition of claim 15, wherein the composition is about 5-15% fibrillar collagen, about 5-20% methylcellulose, about 0.5-10% Bioglass, and about 60-90% carbonate apatite.

27. The composition of claim 14, wherein the methylcellulose has a viscosity of between 5,000 cPs and 15,000 cPs.
